(19) United States
(12) Patent Application Publication
(10) Pub. No.: US 2005/0054720 A1
(43) Pub. Date: Mar. 10, 2005

(54) Saururus cernuus compounds that inhibit cellular responses to hypoxia

(76) Inventors: Dale G. Nagle, Oxford, MS (US); Yu-Dong Zhou, Oxford, MS (US); Chowdhury Faiz Hossain, University, MS (US); Tyler W. Hodges, Tuscaloosa, AL (US)

Correspondence Address: Richard S. Myers, Jr.;Stites & Harbison PLLC, Suite 1800, 424 Church Street, Nashville, TN, 37219-2376, US

(21) Appl. No.: 10/842,177

(22) Filed: May 10, 2004

Related U.S. Application Data
(60) Provisional application No. 60/468,896, filed on May 8, 2003.

(51) Int. Cl. A61K 31/41; C07D 45/02

(52) U.S. Cl. 514/464; 549/435

(57) ABSTRACT

Compounds and compositions that effectively block hypoxia-inducible factor-1 function, and methods of use thereof. The compounds and compositions of the present invention are useful in the prevention and treatment of cancer, stroke, heart disease, ocular neovascular diseases, and arthritis.

### PRIORITY INFORMATION

This application claims priority to U.S. Patent Application No. 60/468,896, filed May 8, 2003, the contents of which are incorporated herein by reference.

### GOVERNMENT SUPPORT

This invention was made with support from Grant Number DAMD 17-01-0566 from the Department of Defense. The Government has rights to this invention.

### FIELD OF THE INVENTION

The present invention relates generally to the field of compounds and compositions that exhibit the ability to inhibit hypoxia-inducible factor-1 function. The present invention also relates to methods of inhibiting hypoxia-inducible factor-1 function. Specific inhibitors of HIF-1 can be useful for the prevention and treatment of cancer, stroke, heart disease, ocular neovascular diseases, psoriasis and arthritis. The compounds of the present invention also inhibit vascular endothelial growth factor.

### BACKGROUND OF THE INVENTION

Gene regulation (selective activation and inactivation of genes) plays an important role in the development and progression of cancer, an assemblage of diseases that result from multiple accumulated mutations. The past two decades have witnessed the rapid expansion of our knowledge of cancer genetics, from a handful of oncogenes to the identification of many genes that affect tumorigenesis, tumor growth, progression, metastasis, and tumor cell death. Elucidation of the molecular mechanisms underlying these events provides the opportunity to develop new mechanism-based therapeutics. As a result, the first molecular targeted agent (Trastuzumab) is in clinical use, and many mechanism-based agents are in clinical trial.

An embodiment of the present invention is the discovery and characterization of potential chemotherapeutic agents that specifically target tumor hypoxia. The existence of hypoxic regions is a common feature of solid tumors. Unlike normal cells from the same tissue, tumor cells are often chronically hypoxic. The extent of tumor hypoxia correlates with advanced stages and poor prognosis. Rapid growth of tumors outstrips the capability of existing blood vessels to supply oxygen and nutrients, and remove metabolic waste. Hypoxia triggers tumor angiogenesis and the newly formed tumor blood vessels often fail to mature. As a result, certain tumor regions are constantly under hypoxic stress due to sluggish and irregular blood flow. Hypoxic tumor cells are more resistant than normoxic tumor cells to radiation treatment and chemotherapy and these hypoxic cells are considered an important contributor to disease relapse. Currently, the general strategies to overcome tumor hypoxia are: 1) increasing tumor oxygenation by means such as breathing carbogen (95% O₂, 5% CO₂); 2) developing chemical sensitizers to increase the sensitivity of hypoxic cells to radiation; and 3) developing hypoxic cytotoxins that selectively kill hypoxic cells. These approaches target the direct effects of hypoxia—lack of cellular oxygen. Presently, there is only one bioreductive drug (tirapazamine) in clinical trial that selectively kills hypoxic tumor cells. No hypoxic cytotoxins are currently approved. It is clear that tumor hypoxia is an important unmet therapeutic need for cancer treatment and drug discovery efforts should be directed at this target.

The focal point of this drug discovery effort is to target the important indirect effect of hypoxia—induction of genes that promote the adaptation and survival of tumor cells. As a form of stress, hypoxia activates both survival and cell death programs. In oncogenically transformed cells, hypoxia provides a physiological pressure and selects for the cells with diminished apoptotic potential. Hypoxic tumor cells that have adapted to oxygen and nutrient deprivation are associated with a more aggressive phenotype and poor prognosis. The transcription factor that plays a role in hypoxia-induced gene expression is hypoxia-inducible factor-1 (HIF-1), a heterodimer of the bHLH-PAS proteins HIF-1α and HIF-1β/ARNT. HIF-1α is degraded rapidly under normoxic conditions and stabilized under hypoxic conditions, while HIF-1β is constitutively expressed. Upon hypoxic induction and activation, HIF-1 binds to the hypoxia response element (HRE) present in the promoters of target genes and activates transcription. Survival genes activated by HIF-1 can be classified into three major functional groups—(i) those that increase oxygen delivery through enhancing angiogenesis, erythropoiesis, and vasodilatation; (ii) those that decrease oxygen consumption through inducing numerous genes involved in anaerobic metabolism (glucose transporters and glycolytic enzymes); and (iii) growth factors. In addition to hypoxia, other tumor-specific mechanisms that increase HIF-1 activity include the activation of oncogenes (i.e. ras, src, myc, etc.) and the loss of tumor suppressor genes (i.e. PTEN, VHL). The oxygen regulated subunit HIF-1α is overexpressed in common human cancers and their metastases, and is associated with advanced stages in breast cancer. In animal models, deletion of either HIF-1α or HIF-1β blocks hypoxic induction of the genes that are normally induced by hypoxia, and is associated with reduced tumor vascularity and retarded tumor growth. In addition, inhibition of HIF-1 function through blocking the interaction between HIF-1 and the coactivator p300/CBP leads to an attenuation of hypoxia-inducible gene expression, reduction of angiogenesis, and suppression of both breast and colon carcinoma cell-derived tumor growth in vivo. In summary, results from multiple animal models indicate that inhibition of hypoxia-induced gene expression through blocking HIF-1 production/function is associated with significant suppression of tumor growth. Therefore, small molecule specific inhibitors of HIF-1 represent potential chemotherapeutic drugs that will suppress tumor growth, progression, and hypoxia associated treatment resistance by inhibiting hypoxia-induced gene expression.

The source of chemicals for the compounds and methods of the present invention is natural product-rich plant, marine invertebrate, and microbe extracts. Natural products have been a major source of new drugs for centuries and the biochemical diversity offered by natural products is unmatchable by any other approach. Statistics show that over 60% of approved anticancer agents are of natural origin (natural products or synthetic compounds based on natural product models). The directed serendipity of natural product drug discovery, empowered by functional bioassays, continues to play a key role in the discovery of numerous chemotherapeutic agents, often with dissimilar modes of action. In the case of HIF-1 activation, the known small molecule inhibitors can be grouped into the following categories: i) regulators of protein phospohorylation that include genistein, sodium fluoride, PD98059, LY294002, wortmannin, and rapamycin; ii) inhibitors of mitochondrial electron transport that include dipheyleneiodonium chloride (DPI), rotenone, and myxothiazol; iii) carbon monoxide, and nitric oxide; iv) transcription inhibitor actinomycin D and protein synthesis inhibitor cycloheximide; and v) Topo I inhibitor topotecan. Most of these small molecule inhibitors are natural products (genistein, wortmannin, rapamycin, rotenone, myxothiazol, actinomycin D, cycloheximide, and topotecan) or natural product derived synthetic compounds (PD98059 and LY294002). These molecules are also known to regulate a number of cellular signaling or metabolic processes other than the hypoxia signaling pathway. Therefore, these compounds are unlikely to function as specific HIF-1 inhibitors. The present invention identifies and characterizes specific inhibitors of hypoxia-activated gene expression and do not effect normoxic cellular signaling.

### SUMMARY AND OBJECTS OF THE INVENTION

The present inventors have discovered extracts and purified compounds isolated from a wetland plant (Saururus cernuus L.) exhibit the ability to potently and effectively inhibit hypoxia-inducible factor-1 function. This effectively blocks hypoxia-activated tumor cell survival pathways and reduces angiogenic growth factor production tumor cells, including human breast tumor cells. In addition, HIF-1 activation is also associated with ischemic tissue damage, following vascular occlusion due to heart attack and stroke. Therefore, inhibitors of HIF-1 can be useful for the prevention and treatment of cancer, heart disease, and stroke. Further, the compounds and compositions of the present invention may enhance the activity of traditional chemotherapy and radiation treatments for cancer. Recent evidence suggests that HIF-1 inhibitors may be useful for the treatment of arthritis. Inhibitors of vascular endothelial growth factor (VEGF) are of potential utility in the treatment of diabetic retinopathy and age-related macular degeneration. VEGF is regulated by HIF-1 and these S. cernuus compounds inhibit both HIF-1 and VEGF in tumor cell line. Therefore, these compounds and compositions may be useful in the treatment and prevention of diabetic retinopathy and macular degeneration.

No substance that inhibits HIF-1 function is currently available for the treatment of cancer, heart disease, stroke, arthritis, diabetic retinopathy, or macular degeneration. Unlike conventional chemotherapy, selective HIF-1 inhibitors can specifically affect target tissues with a low level of non-selective cytotoxicity. Extracts and compounds isolated from S. cernuus have not previously been reported to have application for use in the treatment of cancer, heart disease, stroke, arthritis, diabetic retinopathy, or macular degeneration. One set of compounds (manassantin A and epi-manassantin A) that occur in this plant and another related species (S. chinensis) has been reported to have antitumor effects. However, many of the active lignans found in S. cernuus (manassantin B, epi-manassantin B, saucemeol A, etc.) are structurally distinct from S. chinensis compounds known to have antitumor activity.

Accordingly, an object of the present invention is to provide compounds or compositions of the compounds of the present invention described herein, or salts thereof, including compounds 1 and 4, below and analogs, stereoisomers, and pharmaceutical salts thereof.

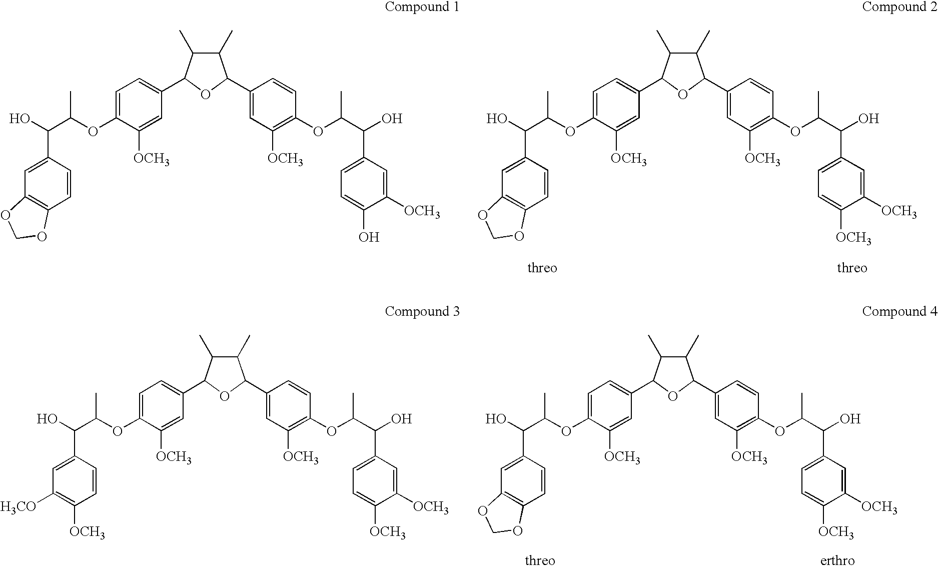

In embodiments of the present invention, these and all other compounds of the present invention are substantially pure. In embodiments, the compounds of the present invention are at least 90% pure. However, the present invention includes mixtures of the compounds of the present invention, crude extracts, and mixtures obtained chromatographically.

Another object of the present invention is to provide methods of inhibiting HIF-1 function by administering to a patient in need thereof a pharmaceutically inhibiting amount of a compound of the present invention, including administration of one of compounds 1-4 above or compound 5, below, in an acceptable pharmaceutical formulation or composition.

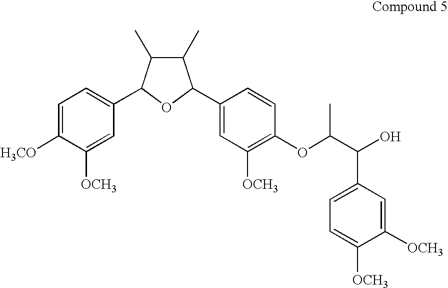

Another embodiment of the present invention is to provide a method of treating cancer comprising administering a cancer treating effective amount of a compound of formula 1, 2, 4, and/or 5, above. The present invention can be used for the treatment of, for example, liver cancer, breast cancer, throat cancer, melanosis, lung cancer, prostate cancer, colon cancer, stomach cancer, cervical cancer, esophageal cancer, tongue cancer, oral cancer, pancreas cancer, thyroid cancer, leukemia and myeloma.

Another object of the present invention is a compound, or a composition comprising a compound of the following Formula I, analogs, stereoisomers, and pharmaceutically acceptable salts thereof: 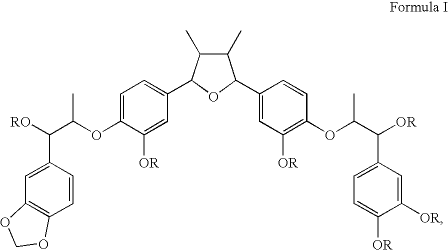
wherein R is the same or different and is H, alkyl, acetyl, amine, amide, cyano, thiocyano, aldehyde, halogen, ester, ether, sulfate, carbonate, acetonide, aldehyde, halides, cyano, thiocyano.

Another object of the present invention is a compound or a composition comprising a compound of the following formula II, analogs, stereoisomers, and pharmaceutically acceptable salts thereof: 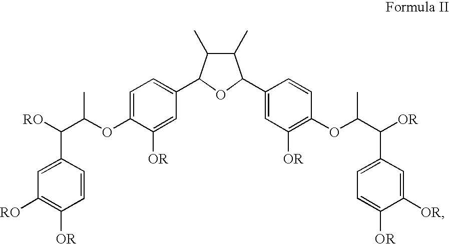
wherein R is the same or different and is H, alkyl, acetyl, amine, amide, cyano, thiocyano, aldehyde, halogen, ester, ether, sulfate, carbonate, acetonide, aldehyde, halides, cyano, thiocyano.

Another object of the present invention is a compound or a composition comprising a compound of the following formula III, analogs, stereoisomers, and pharmaceutically acceptable salts thereof: 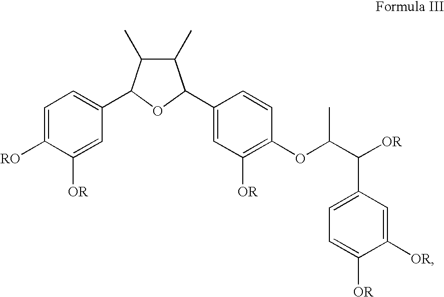
wherein R is the same or different and is H, alkyl, acetyl, amine, amide, cyano, thiocyano, aldehyde, halogen, ester, ether, sulfate, carbonate, acetonide, aldehyde, halides, cyano, thiocyano.

Yet another object of the present invention is a method of inhibiting HIF-1 comprising administering a HIF-1 inhibiting amount of a compound of the present invention or a derivative thereof to a subject in need of such treatment. The compound may be administered as part of a formulation suitable for oral or non-oral administration. A pharmaceutical composition may be formed with the compounds of the present invention in the same manner that the compositions of Hahm et al., WO 01/87869, incorporated herein by reference, are prepared.

Another object of the present invention is a method of treating cancer, heart disease, stroke, chronic inflammatory diseases, arthritis, diabetic retinopathy, or macular degeneration, comprising administering an effective amount of a compound of the present invention, its derivative, or a pharmaceutically acceptable salt thereof.

Another object of the present invention is a method of treating ischemic tissue damage comprising administering an effective amount of a compound of the present invention, its derivative, or a pharmaceutically acceptable salt thereof.

Another object of the present invention is a method of inhibiting vascular endothelial growth factor (VEGF), comprising administering an effective amount of a compound of the present invention, its derivative, or a pharmaceutically acceptable salt thereof.

Other embodiments will be apparent upon a review of the specification and claims.

### BRIEF DESCRIPTIONS OF THE FIGURES

FIG. 1 is a graph showing data related to HIF-1 activation in breast cancer cell lines.

FIG. 2 is a graph showing data related to an isoflavone and a flavonoid derivative for inhibition of hypoxia-induced HIF-1 activation.

FIG. 3 is a graph showing data related to compounds of the present invention for inhibition of hypoxia-induced HIF-1 activation.

FIG. 4 is a graph showing data related to compounds of the present invention for inhibition of hypoxia-induced increase in secreted VEGF protein.

FIG. 5 is a graph showing data related to compounds of the present invention on 1,10-phenanthroline-induced HIF-1 activation.

FIG. 6 is a graph showing data related to compounds of the present invention on −1,10-phenanthroline-induced increase in secreted VEGF protein.

FIG. 7 shows data related to compound 2 for inhibition of hypoxia-induced accumulation of the HIF-1α subunit.

FIG. 8 is a graph showing data related to compounds of the present invention on hypoxia-induced HIF-1 activation (A) and 1,10-phenanthroline-induced HIF-1 activation (B).

FIG. 9 is a graph showing data related to compounds of the present invention for inhibition of hypoxia-induced increase in secreted VEGF protein.

FIG. 10 shows data related to compounds of the present invention for inhibition of hypoxia-induced accumulation of the HIF-1α subunit.

FIG. 11 shows dose response curves of compound 2 for inhibition of cell proliferation in the NCI 60 cell lines.

FIG. 12 shows mean graphs for activity against cancer cell lines for compound 2.

### DETAILED DESCRIPTION OF THE INVENTION

As stated above, embodiment of the present invention include compounds selected from formulae I, II, III, above. Other embodiments include methods of using compounds selected from formulae I, II, III, above.

When used herein with respect to a R group, the term alkyl or alkyl group is to be understood in the broadest sense to mean hydrocarbon residues which can be linear, i.e., straight-chain, or branched, and can be acyclic or cyclic residues or comprise any combination of acyclic and cyclic subunits. Further, the term alkyl as used herein expressly includes saturated groups as well as unsaturated groups which latter groups contain one or more, for example, one, two, or three, double bonds and/or triple bonds. Examples of alkyl residues containing from 1 to 20 carbon atoms are methyl, ethyl, propyl, butyl, pentyl, hexyl, heptyl, octyl, nonyl, decyl, undecyl, dodecyl, tetradecyl, hexadecyl, octadecyl, and eicosyl, the n-isomers of all these residues, isopropyl, isobutyl, 1-methylbutyl, isopentyl, neopentyl, 2,2-dimethylbutyl, 2-methylpentyl, 3-methylpentyl, isohexyl, 2,3,4-trimethylhexyl, isodecyl, sec-butyl, tert-butyl, or tert-pentyl.

Unsaturated alkyl residues are, for example, alkenyl residues such as vinyl, 1-propenyl, 2-propenyl (=allyl), 2-butenyl, 3-butenyl, 2-methyl-2-butenyl, 3-methyl-2-butenyl, 5-hexenyl, or 1,3-pentadienyl, or alkynyl residues such as ethynyl, 1-propynyl, 2-propynyl (=propargyl), or 2-butynyl.

The alkyl groups in general can be substituted or unsubstituted by one or more identical or different substituents. Any kind of substituents can be present in any desired position, including the other R group variables, provided that the substitution does not lead to an unstable molecule. Alkyl residues can also be unsaturated when they are substituted.

All the compounds disclosed herein can be in the form of a composition, such as a pharmaceutical composition. That is, they may be made into drug form for oral or non-oral administration. The present invention accordingly provides a pharmaceutical composition which comprises a compound of this invention in combination or association with a pharmaceutically acceptable carrier. In particular, the present invention provides a pharmaceutical composition which comprises an effective amount of a compound of this invention and a pharmaceutically acceptable carrier.

The term “pharmaceutically acceptable salt” as used herein is intended to include the non-toxic acid addition salts with inorganic or organic acids, e.g. salts with acids such as hydrochloric, phosphoric, sulfuric, maleic, acetic, citric, succinic, benzoic, fumaric, mandelic, p-toluene-sulfonic, methanesulfonic, ascorbic, lactic, gluconic, trifluoroacetic, hydroiodic, hydrobromic, and the like. Examples of pharmaceutically acceptable salts include, but are not limited to, mineral or organic acid salts of basic residues such as amines; alkali or organic salts of acidic residues such as carboxylic acids; and the like.

Pharmaceutically acceptable salts of the compounds of the invention can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

For the extraction of the present active compounds, the procedure as set forth in WO 01/87869 and Rao, U.S. Pat. No. 4,619,943 may be used. Accordingly, in Rao, a variety of solvents may be used either singly or in combination with each other. Suitable solvents include, for example, hydrocarbons, alcohols, ethers, halohydrocarbons, ketones, esters, water and mixtures thereof. Hydrocarbon solvents include aromatic hydrocarbons such as benzene, toluene, and the xylenes, as well as aliphatic and cycloaliphatic hydrocarbons, preferably of 5 to 8 carbon atoms, such as pentane, hexane, heptane, and octane, isomers thereof, and the corresponding cyclic materials, for example, cyclohexane. Ether solvents include aliphatic ethers such as diethyl ether and cyclic ethers such as tetrahydrofuran. Halohydrocarbon solvents may be haloalkanes such as methylene chloride or halophenyl compounds such as chlorobenzene. Ketone solvents will usually be aliphatic ketones of 3 to 6 carbon atoms, for example, acetone or ethyl methyl ketone, or a cycloaliphatic ketone such as cyclopentanone or cyclohexanone. Aliphatic ester solvents such as ethyl or methyl acetate and alcohol solvents, frequently of 1 to 4 carbon atoms, are also useful. Extraction may be by batch or continuous process or vapor-phase method at ambient or elevated temperatures.

In the preferred method set forth by Rao, using 95% ethanol, three extractions at room temperature by batch percolation are carried out, each extraction lasting for two days. The combined extracts are concentrated by using heat, preferably under reduced pressure, to a thick syrup. The viscous concentrate is partitioned between water in the pH range of 2.0-10.0 and a water-immiscible solvent, preferably, ethyl acetate, chloroform, benzene, or ether, whereby the active materials (the SC-neolignans) passes into the organic layer. The extraction and partitioning process provides a solution of substantially free of extraneous plant materials such as water-soluble materials and materials that are insoluble in the water-immiscible partitioning solvent. The water-immiscible solvent layer is concentrated and processed by chromatography to obtain a crude mixture of the active components before final chromatography. In the column chromatography procedure, the concentrate from the partitioning above is taken up in a suitable solvent such as benzene and added to a column made up of silica gel. Other solvents and solvent combinations including hexane, chloroform, toluene, and the like may be used, and adsorbents such as alumina or a magnesium silicate such as Florisil® (a complex magnesium silicate available through Floridin Co., Berkeley Springs, W V 25411) are also suitable. Gross separation into three groups of different polarity is achieved by elution with solvents of increasing polarity, preferably by using benzene, 5-25% acetone in benzene, and 1-10% methanol in benzene. The neuroleptic activity is generally found in the fraction of intermediate polarity after concentration to dryness. Alternatively, a series of partitions between different pairs of solvents may be carried out to achieve a gross separation into fractions of varying polarity.

For further purification and separation of the active compounds, adsorption chromatography, preferably using silica gel, is employed in which the mixture is added as a solution in benzene in a proportion of 15-35 g of adsorbent per gram of mixture in a column of suitable size. Other adsorbents commonly used in chromatography including alumina (acidic, neutral or basic), magnesium silicate such as Florisil, partially etherified cross-linked dextran such as Sephadex® (Pharmacia Fine Chemicals, Piscataway, N.J. 08854), and polyamide are also satisfactory. The column is eluted with benzene, followed by increasing concentrations of acetone (0-25%) in benzene which can be made in discrete steps or in a gradient fashion. Fractions of suitable volume are collected, tested by UV adsorption intensity at 280 nm and thin-layer chromatography (silica gel plates, 5-25% acetone/benzene, visualization: UV light or spray with 1% sulfuric acid and heat to generate crimson red spots), combined based on their composition, and concentrated to dryness. Alternatively, partition chromatography using Sephadex LH20 as the support with 50-80% methanol in water as the stationary phase and ligroin with a 0-75% benzene gradient as the mobile phase may be used to effect this further separation into groups based on polarity.

For final purification, high performance liquid chromatography may be used for obtaining biologically pure individual components. A variety of commercially available column packings, with eluting solvents such as aliphatic/aromatic hydrocarbons, halohydrocarbons, lower alcohols, or lower aliphatic ketones may be employed at pressures ranging from 0-5000 p.s.i. for an efficient separation of the individual components in chemical and biological purity.

The method of the present invention includes administering the effective compounds described herein to people or animals by any route appropriate to the condition to be treated, as determined by one of ordinary skill in the art. Additionally, physiologically acceptable acid addition salts of compounds described herein are also useful in the methods of treating of the present invention. Additionally, where appropriate, methods of the present invention include administering the crude extract directly, without the need to be combined with a pharmaceutical composition. For example, the crude extract may be administered to inhibit HIF1 activity in a subject. Examples of methods of administering the crude extract include oral dosages, creams, eye drops, etc.

For other embodiments, the compounds described herein may be taken up in pharmaceutically acceptable carriers, such as, for example, solutions, suspensions, tablets, capsules, ointments, elixirs and injectable compositions. Pharmaceutical preparations may contain from 0.1% to 99% by weight of active ingredient. Preparations which are in single dose form, “unit dosage form”, preferably contain from 20% to 90% active ingredient, and preparations which are not in single dose form preferably contain from 5% to 20% active ingredient. As used herein, the term “active ingredient” refers to compounds described herein, salts thereof, and mixtures of compounds described herein with other pharmaceutically active compounds. Dosage unit forms such as, for example, tablets or capsules, typically contain from about 0.05 to about 1.0 g of active ingredient.

Suitable routes of administering the pharmaceutical preparations include, for example, oral, rectal, eye drops, topical (including dermal, buccal and sublingual), vaginal, parenteral (including subcutaneous, intramuscular, intravenous, intradermal, intrathecal and epidural) and by naso-gastric tube. It will be understood by those skilled in the art that the preferred route of administration will depend upon the condition being treated and may vary with factors such as the condition of the recipient.

According to the methods of the present invention, the effective compounds described herein may be administered alone or in conjunction with other pharmaceutically active compounds. It will be understood by those skilled in the art that pharmaceutically active compounds to be used in combination with the compounds described herein will be selected in order to avoid adverse effects on the recipient or undesirable interactions between the compounds. As used herein, the term “active ingredient” is meant to include compounds described herein when used alone or in combination with one or more additional pharmaceutically active compounds. The amount of the compounds described herein required for use in the various treatments of the present invention depend, inter alia, on the route of administration, the age and weight of the animal (e.g. human) to be treated and the severity of the condition being treated.

The compounds of the present invention may be administered as pharmaceutical formulations. Useful formulations comprise one or more active ingredients and one or more pharmaceutically acceptable carriers. The term “pharmaceutically acceptable” means compatible with the other ingredients of the formulation and not toxic to the recipient. Useful pharmaceutical formulations include those suitable for oral, rectal, nasal, topical, vaginal or parenteral administration, as well as administration by naso-gastric tube. The formulations may conveniently be prepared in unit dosage form and may be prepared by any method known in the art of pharmacy. Such methods include the step of bringing the active ingredient into association with the carrier, which may constitute one or more accessory ingredients. In general, the formulations are prepared by uniformly bringing the active ingredients into association with liquid carriers or finely divided solid carriers or both, and then, if necessary, shaping the product.

### High-Throughput Bioassay for Inhibitors of HIF-1 Activation

Hypoxia-regulated gene expression (selective activation and inactivation of genes) plays an important role in tumor cell adaptation to hypoxia and overall treatment resistance. The transcription factor HIF-1 is a key regulator of hypoxia-regulated gene expression. Compounds that can specifically regulate HIF-1 represent potential drug leads that will target tumor hypoxia and have little effect on well-oxygenated normal cells. Discovery efforts directed at finding specific functional antagonists of HIF-1 can lead to the identification of selective hypoxia/HIF-1 pathway inhibitors.

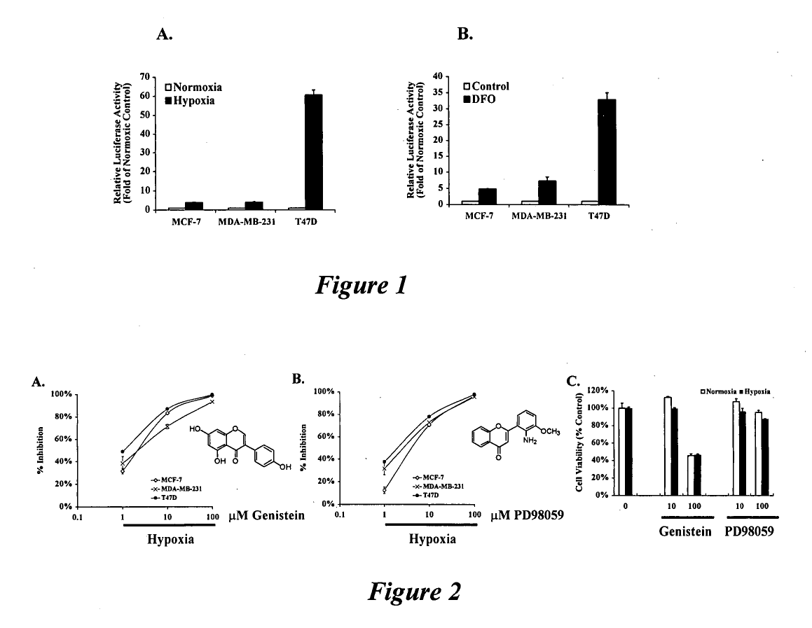

To identify functional antagonists of HIF-1, the present inventors have established a cell-based reporter assay for inhibitors of HIF-1 in hypoxia responsive human breast carcinoma T47D cells. Breast cancer was chosen as an example target for this drug discovery effort, due to the high incidence of this disease and the urgent need to identify chemotherapeutic agents that target tumor hypoxia, the comprehensive knowledge base of breast cancer etiology, and the availability of well-studied human breast carcinoma cell lines as in vitro models. In addition, HIF-1α overexpression is associated with advanced stages of breast cancer and poor prognosis. The activity of HIF-1 is monitored using a luciferase reporter under the control of HRE from the erythropoietin gene (pTK-HRE3-luc, described in reference 31). Exponentially grown T47D cells are transiently transfected with the pTK-HRE3-luc reporter by electroporation and plated into 96-well plates. After 24 hr, cells are exposed to hypoxic conditions (1% O₂/5% CO₂/94% N₂ or chemical hypoxia, iron chelator desferrioxamine (DFO) at 100 μM) for 16 hr. The cells are then lysed and luciferase activity measured. Unless otherwise specified, the low oxygen hypoxic set of conditions (1% O₂/5% CO₂/94% N₂) is referred to simply as hypoxia. Similar analysis are also performed in the human breast carcinoma cell lines MCF-7 and MDA-MB-231. The data from three cell lines is shown below. As shown in FIG. 1A, hypoxic exposure activates HIF-1 in all three cell lines and the most robust response is observed in T47D cells (60-fold induction). HIF-1 activation by the hypoxia mimetic DFO is observed in cell lines under normoxic conditions, and the strongest response is observed in T47D cells (B).

Although it has been shown that the flavonoids genistein and PD98059 can inhibit HIF-1 activation, no quantitative dose-response studies to determine IC₅₀ values have been published. Therefore, the present inventors have established the IC₅₀ of genistein and PD98059 for inhibition of hypoxia-induced HIF-1 activation in MCF-7, MDA-MB-231 and T47D cells.

Test compounds are added to the pTK-HRE3-luc transfected cells at the final concentrations of 1, 10, and 100 μM. Following incubating at 37° C. for 30 min, the cells are exposed to hypoxic conditions for 16 hr, lysed, and luciferase activity measured. The data is presented in FIG. 2 (genistein-2A, PD98059-2B). The structures of each are shown on the right of each figure. In T47D cells, the IC₅₀ for genistein is 1 μM and PD98059 is 2 μM. Under normoxic and hypoxic conditions, PD98059 exerts no significant cytotoxicity at the highest concentration tested, while 100 μM genistein treatment leads to a 50% reduction of T47D cell viability (C). As discussed earlier, genistein is a broad spectrum inhibitor of protein tyrosine kinases. The observed cytotoxicity is most likely caused by nonspecific inhibition of signaling pathways that require tyrosine kinases. Therefore, as a means to dereplicate nonspecific inhibitors of intracellular signaling pathways, the effects of active extracts on cell viability is examined as one of the secondary bioassays.

### Evaluation of Natural Product Extracts for Inhibitors of HIF-1 Activation

The present inventors take a natural product chemical approach that uses the HTS assay for HIF-1 functional antagonists as the primary assay to identify new specific inhibitors of HIF-1 activation. Natural product-rich extracts (dissolved in DMSO) are tested at the final concentration of 5 μg/ml. Crude extracts that can inhibit hypoxic activation of HIF-1 by at least 70% (equivalent to 30% activity of the solvent control) are considered active. If we assume that the active compound constitutes 5% of the extract (which is often lower) and the molecular weight is 500, then the concentration of the active compound for 70% inhibition will be 0.5 μM, 1/20 of that for genistein or PD98059 (10 μM).

Chemical dereplication of extracts that contain hypoxia-selective HIF-1 inhibitors will be achieved through chromatographic separation by HPLC and analysis by UV (UV-photodiode array) and mass spectrum (LC-ESIMS). Several distinct sets of lignans and other substances have been clearly distinguished by LC-ESIMS using a ThermoFinnigan aQa thermoquest system (NCNPR, UM).

### Identification and Molecular Characterization of Potent Natural Product Inhibitors that Selectively Inhibit Hypoxic Activation of HIF-1

The chemically-rich active extract from the aquatic plant Saururus cernuus L. (Saururaceae) is subjected to bioassay-guided chromatographic fractionation. Active fractions are further purified by a combination of normal phase and reversed phase HPLC. Several series of highly potent HIF-1 inhibitory lignans/neolignans are isolated and their structures determined spectroscopically/spectrometrically.

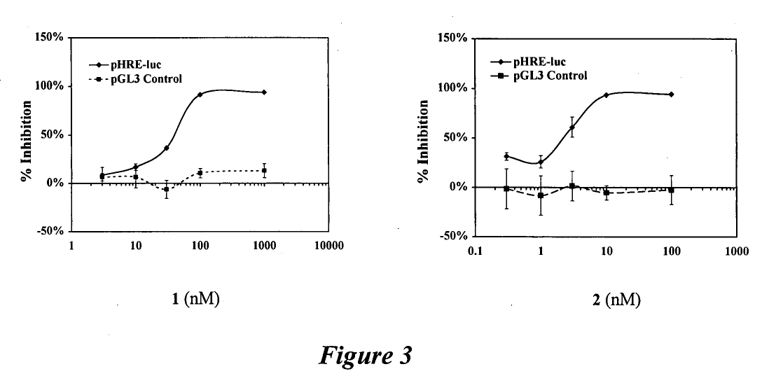

The chemical structures of active compounds 1 and 2 are shown above. Compound 2 is the known compound manassantin B and 1 is a novel compound of the present invention. Both compounds inhibit hypoxic activation of HIF-1 in the T47D cell-based reporter assay (FIG. 3). Data shown are means from one experiment performed in triplicate and the bars represent mean standard deviation. Similar results are obtained from separate experiments. The IC₅₀ is 3 nM for 2 and 30 nM for 1. Complete inhibition is observed at 10 nM for 2 and 100 nM for 1. ANOVA analysis reveals that these inhibitory activities are statistically significant, relative to hypoxic control (p<0.0001). No statistically significant difference is observed on luciferase expression from the control plasmid (pGL3 Control) in the presence of either compound, indicating that the observed inhibition is specific for HIF-1.

As a master regulator of oxygen homeostasis, HIF-1 regulates the expression of many genes that promote cell survival and adaptation to hypoxia. One such HIF-1 target gene is VEGF, an important pro-angiogenic factor secreted by tumor cells to promote new blood vessel formation. Among cancer patients, increased VEGF protein level correlates with high microvessel density, advanced stage disease, and poor prognosis. Inhibitors of VEGF production/function are currently in clinical trials for cancer. Since secreted VEGF protein is the bioactive form, compounds that can reduce the level of secreted VEGF protein represent potential tumor angiogenesis inhibitors. We reason that compounds that can inhibit both hypoxic activation of HIF-1 and hypoxic induction of secreted VEGF protein represent “true” leads that target tumor hypoxia.

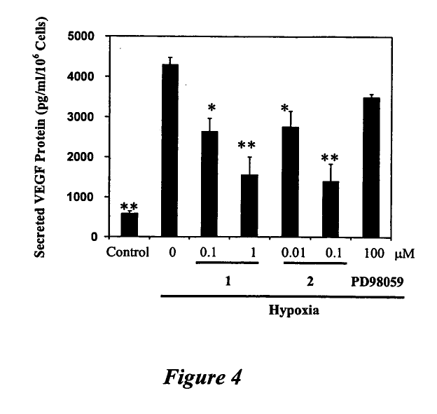

The effects of compounds 1 and 2 on hypoxic induction of secreted VEGF protein are examined in T47D cells. Exponentially grown T47D cells are plated at the density of 30,000 cells/well into 96-well plates. Compound treatment and hypoxic exposure are the same as described. Following incubation, the concentration of secreted VEGF protein in the conditioned media is determined by ELISA (R & D Systems) and the data normalized by the number of viable cells. Results shown in FIG. 4 are averages from a representative experiment performed in quadruplicate, the bars represent standard error. An asterisk (*) indicates the level of significance (p<0.01) relative to the hypoxic control, two asterisks (**) p<0.0001), and no asterisk indicates no statistical difference (ANOVA and Fisher's PLSD post hoc test). Although 1 (at 100 nM), 2 (at 10 nM), and PD98059 (at 100 μM) all completely inhibit hypoxic activation of HIF-1 in the pHRE-luc assay, only 1 and 2 treatments lead to statistically significant reduction of secreted VEGF protein induced by hypoxia. The results on 1 and 2 are similar to those observed in the pHRE-luc assay, where 2 is ten times more potent than 1 in reducing the level of secreted VEGF protein. The ELISA assay with recombinant VEGF protein standard and compounds 1 and 2 reveal that neither compound interferes with the ELISA assay, indicating that the observed inhibition is due to a bonafide reduction of secreted VEGF protein.

Iron chelators and transition metals (such as cobalt and nickel) can activate HIF-1 and have been used as chemical hypoxia mimetics. We have found that the Fe²⁺ selective chelator 1,10-phenanthroline is at least ten times more potent than the commonly used Fe³⁺ selective chelator DFO in activating HIF-1. The expression of the HIF-1α subunit quantitatively determines HIF-1 biological activity (activation of transcription). Additionally, the present inventors found that 1,10-phenanthroline (at 100 μM) is a stronger inducer of the oxygen regulated HIF-1α subunit protein, relative to DFO at 100 μM. Therefore, we have been using 1,10-phenanthroline (10 μM) to induce chemical hypoxia in our system.

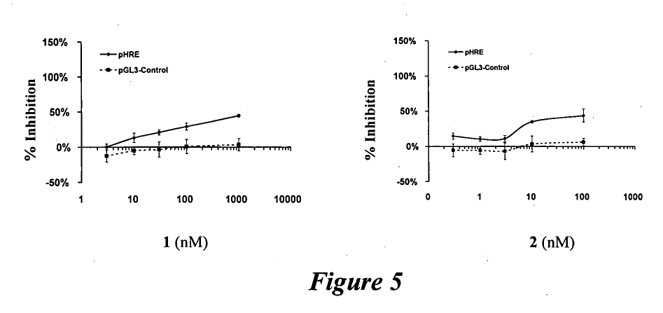

The effects of compounds 1 and 2 on HIF-1 activation by 1,10-phenanthroline are examined and the data shown in FIG. 5. Significantly higher concentrations of both compounds are required to inhibit HIF-1 activation by 1,10-phenathroline (45% inhibition with 1 μM 1 and 44% with 100 nM 2). Neither compound exerts significant effect on luciferase expression from the pGL3-Control plasmid. If we define a specificity index (SI) as SI=[IC₅₀ for pTK-HRE3-luc by 1,10-phenanthroline]/[IC₅₀ for pTK-HRE3-luc by hypoxia], then 1 has a SI>33 and 2 SI>33. These results suggest that compounds 1 and 2 are highly selective inhibitors of HIF-1 activation by physiological hypoxia (reduction in oxygen tension). To our knowledge, these are the first highly potent hypoxia-selective inhibitors of HIF-1 activation.

Recently, Rapisardra et al. identified three camptothecin analogues and one quinocarmycin analogue as inhibitors of HIF-1 activation from a collection of approximately 2,000 pure compounds representing the maximal three-dimensional chemical diversity in the representative NCI compound library, using a comparable HTS reporter assay. The best characterized active compound, topotecan, inhibits both hypoxia-activated and iron chelator (DFO)-activated HIF-1 (EC₅₀: 71.3 nM for hypoxia, and 181 nM for DFO) in U251 human glioma cells. Topotecan is also a DNA topoisomerase I inhibitor and has been used clinically as an antineoplastic agent for cancer. If we use the SI=[EC₅₀ for DFO]/[EC₅₀ for hypoxia] formula, then topotecan has a SI of 2.5. This suggests that HIF-1 inhibitors such as topotecan may have only limited selectivity towards physiological hypoxia, and also target a process (or target) common to HIF-1 activation by both hypoxia and iron chelators.

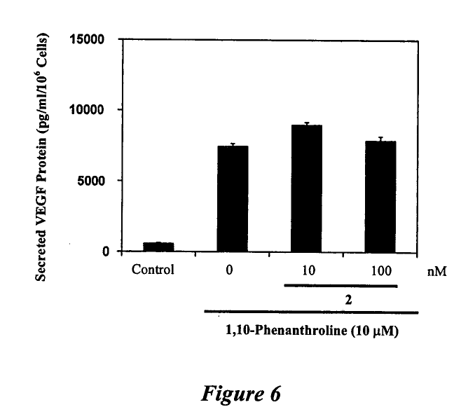

The present inventors further examine the impact of compound 2 on the induction of secreted VEGF protein by 1,10-phenanthroline. Cell plating and compound treatment is similar to those described earlier for hypoxic conditions. Following the test compound treatment for 30 minutes, 1,10-phenanthroline (10 μM final) is added and the incubation continued for another 16 hr at 37° C. At the end of the incubation, the concentration of secreted VEGF protein in the conditioned media is determined by ELISA and the data normalized by the number of viable cells. As shown in FIG. 6, 1,10-phenanthroline treatment significantly induces secreted VEGF protein level in T47D cells, and compound 2 does not effect the induction of secreted VEGF protein by 1,10-phenanthroline.

Since the biological activity of HIF-1 is determined by the availability of HIF-1α protein and hypoxia induces HIF-1α protein, the present inventors examined the effect of compound 2 treatment on the induction of HIF-1α protein.

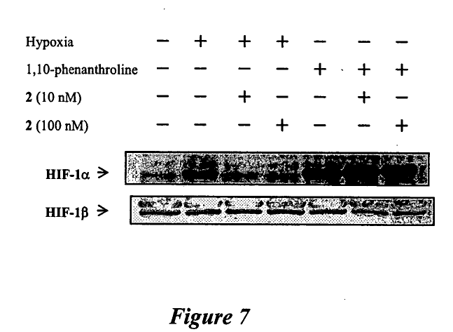

Due to the low abundance of HIF-1α protein in whole cell extract, nuclear extract is prepared and the level of HIF-1α protein in the nuclear extract determined by Western blot. Briefly, exponentially grown T47D cells are exposed to compound 2 for 30 minutes. The incubation then continues for another 4 hr at 37° C. in the presence of either 10 μM 1,10-phenanthroline or hypoxia. Nuclear extracts are prepared from control and treated cells, separated on a SDS/PAGE gel, the separated proteins transferred to a nitrocellulose membrane, incubated with the primary antibodies (anti-HIF-1 α, and anti-HIF-1β monoclonal antibodies, Novus Biologicals), secondary antibody biotinylated anti-mouse immunoglobulin G and Vectastain ABC reagent (Vector Laboratories), and developed using ECL reagents (Amersham Biosciences). As shown in FIG. 7, both hypoxia and 1,10-phenanthroline induce nuclear HIF-1 a protein and exert no significant effect on the constitutively expressed HIF-1 protein. Compound 2 specifically inhibits the hypoxic induction of HIF-1α protein, but not 1,10-phenanthroline induced HIF-1α protein. No change in HIF-1β protein level is observed across the treatments. It is reasonable to conclude that the selectivity of compound 2 towards hypoxia-activated HIF-1 is caused by selective blockade of the hypoxic induction of HIF-1α protein.

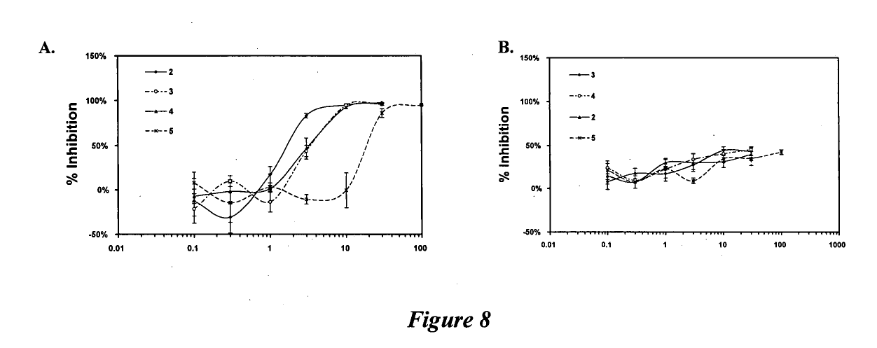

In addition to compounds 1 and 2, the present inventors also isolated 18 structurally related pure compounds from active fractions of the Saururus cernuus extract. Out of the eighteen compounds, four inhibit HIF-1 activation by greater than 50% at 0.01 ppm, one at 0.1 ppm, five at 1 ppm, and eight are inactive. Included in the isolated compounds are the four most active ones (2, 3, 4, 5). Initial studies reveal that compounds 2, 3, and 4 completely inhibited hypoxic activation of HIF-1 at 10 nM, 5 at 100 nM, and the other compounds show no greater than 50% inhibition at concentrations up to 1 μM. Further dose-response studies are performed on the four most active compounds and the data are presented in FIG. 8. T47D cells transfected with the pTK-HRE3-luc reporter and the control plasmid pRL-TK (Promega) are plated into 96-well plates, incubated with test compounds at the concentrations indicated for 30 minutes, followed by hypoxic incubation (FIG. 8A) or exposure to 10 μM 1,10-phenanthroline (FIG. 8B) for another 16 hr. At the end of the incubation, the cells are lysed, luciferase (from pTK-HRE3-luc) and Renilla luciferase (from pRL-TK) activities determined using a Dual-Luciferase® Reporter Assay System (Promega). The luciferase data are normalized with the internal control Renilla luciferase and are presented as average from one representative experiment performed in triplicate and the bars represent standard error.

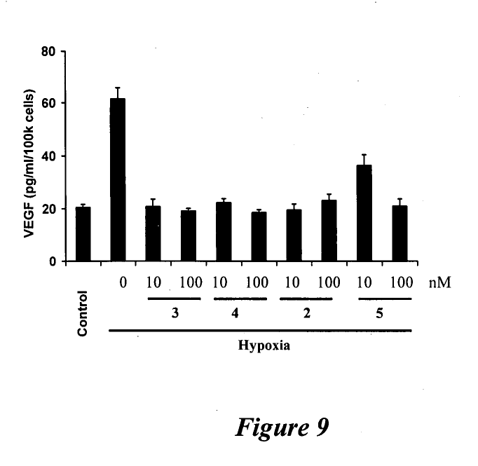

As discussed earlier, one important HIF-1 target gene is VEGF, a key factor for tumor angiogenesis. Compounds that can inhibit both HIF-1 activation and the production of secreted VEGF protein represent potential efficacious chemotherapeutic agents that inhibit both tumor survival and hypoxia induced angiogenesis. The effects of four active examples (compounds 2, 3, 4, and 5) on hypoxic induction of secreted VEGF protein are examined in T47D cells and the data are presented in FIG. 9. Exponentially grown T47D cells are plated at the density of 356,000 cells/well into 12-well plates. Compound treatment and hypoxic exposure are the same as described. Following incubation, the concentration of secreted VEGF protein in the conditioned media is determined by ELISA (R & D Systems) and the data normalized by the number of viable cells. Results shown in FIG. 9 are averages from a representative experiment performed in triplicate, and the bars represent standard error. Hypoxic exposure leads to a 2-fold increase in the level of secreted VEGF protein. Compounds 2, 3, 4, and 5 all significantly inhibit VEGF protein levels relative to the hypoxia-induced positive control. The level of significance (p<0.01) is determined by ANOVA and Fisher's PLSD post hoc test. The exemplary compounds 2, 3, and 4 all completely block hypoxic induction of secreted VEGF protein at concentrations as low as 10 nM. The exemplary compound 5 inhibits the hypoxic induction by 60% at 10 nM, and completely blocks at 100 nM. When tested at 100 nM, none of the compounds interfer with the ELISA assay using VEGF standard (no greater than 15% difference). These results suggest that the manassantins and active derivatives are potential chemotherapeutic agents that inhibit both tumor angiogenesis and hypoxic survival.

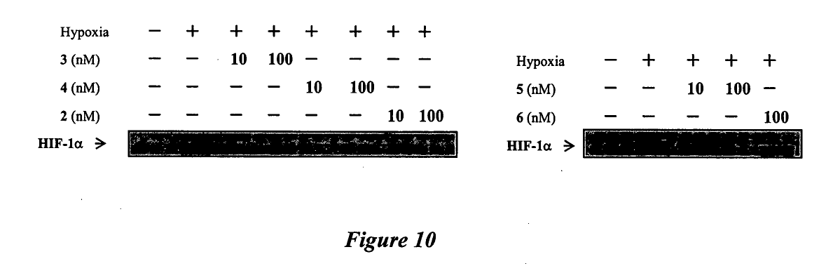

To investigate the mechanism of action, the effects of these compounds on hypoxic stabilization of HIF-1α protein are examined in T47D cells using Western blot. As shown in FIG. 10, all four of the compounds block hypoxia induced stabilization of HIF-1α protein, while the inactive compound 6 has no effect.

In summary, this invention describes the discovery of one class of physiological hypoxia selective inhibitors of HIF-1 activation and the pharmacophores that are required for such function. These compounds represent potential chemotherapeutic agents that can inhibit tumor cell survival, progression, metastasis, and treatment resistance.

Activation of HIF-1 by hypoxia also leads to the activation of cell death genes such as p53, NIP3 and NIX. The activation of these proapoptotic genes is associated with ischemic tissue damage, following vascular occlusion due to heart attack and stroke. The active compounds described herein can also have the potential application of preventing ischemic tissue damage following heart attack and stroke.

Recent research supports the potential application of HIF-1 inhibitors for the treatment and prevention of arthritic conditions such as rheumatoid arthritis. Therefore, crude S. cernuus preparations, and purified compounds 1-5 (or stereoisomers, derivatives, or salts of 1-5) may be useful for the treatment of arthritis.

Ophthalmic complications arising from the pathologic growth of new blood vessels within the eye (ocular neovascularization), are responsible for vision loss in eye diseases that include retinopathy of prematurity (ROP), diabetic retinopathy (PDR), and age-related macular degeneration (AMD). These three ocular neovascular diseases afflict patients in all stages of life—infant, adult, and the elderly, and account for most instances of legal blindness.

Currently, the major procedure to treat vascular diseases of the retina and choroid is surgery. The surgical procedures used to treat vascular diseases of the eye (ROP, PDR, and AMD) are only partially effective, and they introduce significant damage to the retina. Biological processes associated with the retina's attempt to repair itself, such as inflammation, fibrosis, gliosis, scarring, neovascularization, etc., can ultimately lead to partial or complete vision loss. Small drug-like molecules that can inhibit neovascularization within the eye will have the advantage of being safe, effective, inexpensive, and producing fewer side effects.

Recent studies have highlighted vascular endothelial growth factor (VEGF), as the key angiogenic inducer in pathologic ocular neovascularization. The level of VEGF is low (or undetectable) in healthy subjects, and high intraocular VEGF levels correlate with active neovascularization in patients with ischemic disorders, including PDR, proliferative vitreoretinopathy, proliferative sickle cell retinopathy, retinal vein occlusion, and AMD. Intraocular VEGF levels decline to basal level after successful laser photocoagulation therapy, with the cessation of neovascularization.

The pattern of VEGF expression matches that of retinal ischemia, suggesting that VEGF is induced by retinal ischemia to promote intraocular neovascularization. Compounds that inhibit hypoxic induction of VEGF can therefore block intraocular neovascularization induced by retinal ischemia, and have therapeutic potential for ophthalmic complications caused by ocular neovascularization. Therefore, crude S. cernuus preparations, and purified compounds 1-5 (or derivatives of 1-5) may be useful in the treatment and prevention of macular degeneration and other related neovascular disorders of the eye.

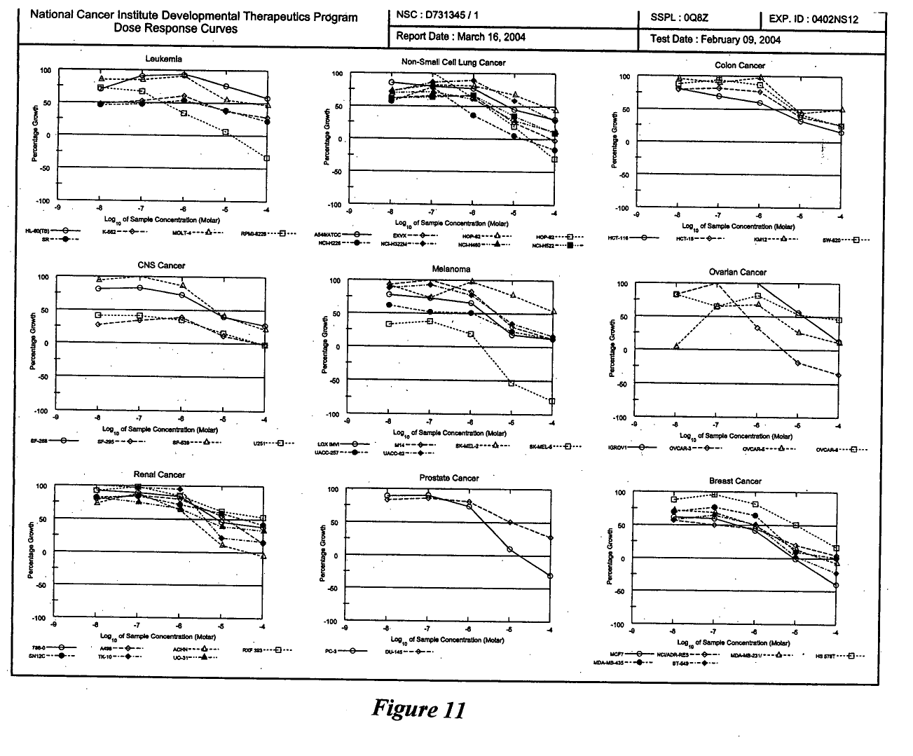

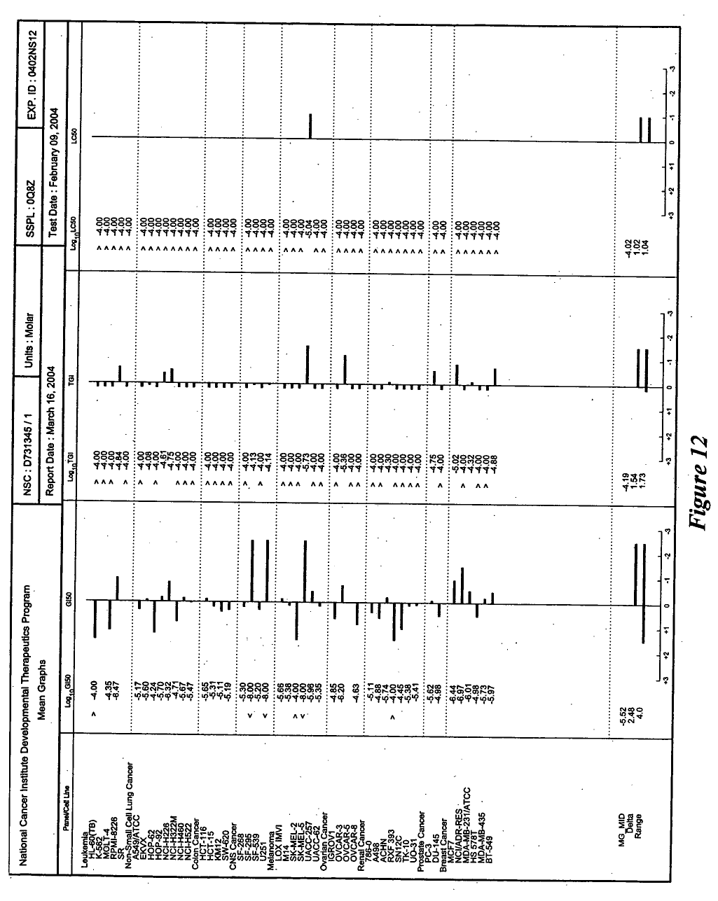

For exemplary purposes, an embodiment of the present invention was tested for activity with respect to certain cancer cell lines. FIGS. 11 and 12 are results from in vitro 60-cell line antitumor testing from the US National Cancer Institute Developmental Therapeutics Program (NCI-DTP) on compound 2. The testing was in connection with the NCI's In Vitro Cell Line Screening Project (IVCLSP). Detailed information regarding the experiments and their interpretation is available from the NCI.

FIG. 11 depicts the Dose Response Curves for percent growth of tumor cell lines, grouped into various categories (such as colon, breast, etc.) The curves represent the response of each tumor cell line when treated with compound 2.

FIG. 12 depicts the GI₅₀, TGI, and LC₅₀ (left to right) Mean Graphs for tumor cell lines, grouped into various categories (such as colon, breast, etc.) The data are arranged to display the relative response selectivity of each cell line relative to one other. This form of data display is used to examine the relative tumor specificity of a text compound. The curves represent the response of each tumor cell line when treated with compound 2.

Antitumor agents with tumor-specific selectivity are highly desired, in order to reduce the incidence of non-selective cytotoxicity to patients. The data shown in these figures clearly indicate that compound 2 is highly selective (up to 10,000-fold in some cases) for tumor cell lines (e.g. two CNS tumor lines, a melanoma cell line, and several breast tumor cell lines). Compound 2 produces a superior and unexpected level of selectivity, as indicated by this GI₅₀ 60-cell line pattern, than that observed for currently approved antitumor drugs. Compound 2 also potently inhibits the NCI/ADR-RES breast tumor cell line. This is of special importance since this NCI/ADR-RES cell line is resistant to many currently used antitumor agents such as adriamycin, taxol, and others.

The invention thus being described, it would be obvious that the same can be varied in many ways. Such variations that would be obvious to one of ordinary skill in the art is to be considered as being part of this disclosure.

Unless otherwise indicated, all numbers expressing quantities of ingredients, properties such as reaction conditions, and so forth used in the Specification and claims are to be understood as being modified in all instances by the term “about.” Accordingly, unless indicated to the contrary, the numerical parameters set forth in the Specification and claims are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

### REFERENCES CITED

Throughout the Specification (including the following list), various patents and/or publications are cited. Each patent/publication (again, specifically including the list below) is incorporated herein by reference in its entirety, and is considered part of this disclosure.
1. Gibbs J B (2000) Mechanism-based target identification and drug discovery in cancer research. Science 287, 1969-1973.
2. Brown J M, Giaccia A J (1998) The unique physiology of solid tumors: opportunities (and problems) for cancer therapy. Cancer Res 58, 1408-1416.
3. Tomida A, Tsuruo T (1999) Drug resistance mediated by cellular stress response to the microenvironment of solid tumors. Anti-Cancer Drug Design 14, 169-177.
4. Brown J M (2000) Hypoxic cytotoxic agents: a new approach to cancer chemotherapy. Drug Resist Updat 3, 7-13.
5. von Pawel J, von Roemeling R, Gatzemeier U, Boyer M, Elisson L O, Clark P, Talbot D, Rey A, Butler T W, Hirsh V, Olver I, Bergman B, Ayoub J, Richardson G, Dunlop D, Arcenas A, Vescio R, Viallet J, Treat J (2000) Tirapazamine plus cisplatin versus cisplatin in advanced non-small-cell lung cancer: A report of the international CATAPULT I study group. Cisplatin and Tirapazamine in Subjects with Advanced Previously Untreated Non-Small-Cell Lung Tumors. J Clin Oncol 18, 1351-1359.
6. Saikumar P, Dong Z, Weinberg J M, Venkatachalam M A (1998) Mechanisms of cell death in hypoxia/reoxygenation injury. Oncogene 17, 3341-3349.
7. Graeber T G, Osmanian C, Jacks T, Housman D E, Koch C J, Lowe S W, Giaccia A J (1996) Hypoxia-mediated selection of cells with diminished apoptotic potential in solid tumours. Nature 379, 88-91.
8. Dachs G U, Tozer G M (2000) Hypoxia modulated gene expression: angiogenesis, metastasis and therapeutic exploitation. Eur J Cancer 36, 1649-1660.
9. Semenza G L (1999) Regulation of mammalian O₂ homeostasis by hypoxia-inducible factor 1. Annu Rev Cell Dev Biol 15, 551-578.
10. Semenza G L (2001) HIF-1, O₂, and the 3 PHDs: How animal cells signal hypoxia to the nucleus. Cell 107, 1-3.
11. Pugh C W, Gleadle J, Maxwell P H (2001) Hypoxia and oxidative stress in breast cancer. Hypoxia signalling pathways. Breast Cancer Res 3, 313-317.
12. Zhong H, De Marzo A M, Laughner E, Lim M, Hilton D A, Zagzag D, Buechler P, Isaacs W B, Semenza G L, Simons J W (1998) Overexpression of hypoxia-inducible factor 1alpha in common human cancers and their metastases. Cancer Res 59, 5830-5835.
13. Bos R, Zhong H, Hanrahan C F, Mommers E C, Semenza G L, Pinedo H M, Abeloff M D, Simons J W, van Diest P J, van der Wall E (2001) Levels of hypoxia-inducible factor-1 alpha during breast carcinogenesis. J Natl Cancer Inst 93, 309-314.
14. Ryan H E, Lo J, Johnson R S (1998) HIF-1α is required for solid tumor formation and embryonic vascularization. EMBO J. 17, 3005-3015.
15. Maxwell P H, Dachs G U, Gleadle J M, Nicholls L G, Harris A L, Stratford I J, Hankinson O, Pugh C W, Ratcliffe P J (1997) Hypoxia-inducible factor-1 modulates gene expression in solid tumors and influences both angiogenesis and tumor growth. Proc Natl Acad Sci USA 94, 8104-8109.
16. Jiang B H, Agani F, Passaniti A, Semenza G L (1997) V-SRC induces expression of hypoxia-inducible factor 1 (HIF-1) and transcription of genes encoding vascular endothelial growth factor and enolase 1: involvement of HIF-1 in tumor progression. Cancer Res 57. 5328-5335.
17. Ryan H E, Poloni M, McNulty W, Elson D, Gassmann M, Arbeit J M, Johnson R S (2000) Hypoxia-inducible factor-1alpha is a positive factor in solid tumor growth. Cancer Res 60, 4010-4015.
18. Kung A L, Wang S, Klco J M, Kaelin W G, Livingston D M (2000) Suppression of tumor growth through disruption of hypoxia-inducible transcription. Nat Med 6, 1335-1340.
19. Cragg G M, Newman D J, Snader K M (1997) Natural Products in Drug Discovery and Development. J Nat Prod 60, 52-60.
20. Wang G L, Jiang B H, Semenza G L (1995) Effect of protein kinase and phosphatase inhibitors on expression of hypoxia-inducible factor 1. Biochem Biophys Res Commun 216, 669-675.
21. Richard D E, Berra E, Gothie E, Roux D, Pouyssegur J (1999) p42/p44 mitogen-activated protein kinases phosphorylate hypoxia-inducible factor 1 alpha (HIF— 1 alpha) and enhance the transcriptional activity of HIF-1. J Biol Chem 274, 32631-32637.
22. Zhong H, Chiles K, Feldser D, Laughner E, Hanrahan C, Georgescu M M, Simons J W, Semenza G L (2000) Modulation of hypoxia-inducible factor 1 expression by the epidermal growth factor/phosphatidylinositol 3-kinase/PTEN/AKT/FRAP pathway in human prostate cancer cells: Implications for tumor angiogenesis and therapeutics. Cancer Res 60, 1541-1545.
23. Jiang B H, Jiang G, Zheng J Z, Lu Z, Hunter T, Vogt P K (2001) Phosphatidylinositol 3-kinase signaling controls levels of hypoxia-inducible factor 1. Cell Growth Differ 12, 363-369.
24. Gleadle J M, Ebert B L, Ratcliffe P J (1995) Diphenylene iodonium inhibits the induction of erythropoietin and other mammalian genes by hypoxia. Implications for the mechanism of oxygen sensing. Eur J Biochem 234, 92-99.
25. Chandel N S, Maltepe E, Goldwasser E, Mathieu C E, Simon M C, Schumacker P T (1998) Mitochondrial reactive oxygen species trigger hypoxia-induced transcription. Proc Natl Acad Sci USA 95, 11715-11720.
26. Liu Y, Christou H, Morita T, Laughner E, Semenza G L, Kourembanas S (1998) Carbon monoxide and nitric oxide suppress the hypoxic induction of vascular endothelial growth factor gene via the 5′ enhancer. J Biol Chem 273, 15257-15262.
27. Huang L E, Willmore W G, Gu J, Goldberg M A, Bunn H F (1999) Inhibition of hypoxia-inducible factor 1 activation by carbon monoxide and nitric oxide. Implications for oxygen sensing and signaling. J Biol Chem 274, 9038-9044.
28. Wang G L, Semenza G L (1993) Characterization of hypoxia-inducible factor 1 and regulation of DNA binding activity by hypoxia. J Biol Chem 1993 268, 21513-21518.
29. Semenza G L, Wang G L (1992) A nuclear factor induced by hypoxia via de novo protein synthesis binds to the human erythropoietin gene enhancer at a site required for transcriptional activation. Mol Cell Biol 12, 5447-5454.
30. Rapisarda A, Uranchimeg B, Scudiero D A, Selby M, Sausville E A, Shoemaker R H, Melillo G (2002) Identification of small molecule inhibitors of hypoxia-inducible factor 1 transcriptional activation pathway. Cancer Res 62, 4316-4324.
31. Tian H, McKnight S L, Russell D W (1997) Endothelial PAS domain protein 1 (EPAS1), a transcription factor selectively expressed in endothelial cells. Genes Dev 11, 72-82.
32. Ferrrara N (1999) Role of vascular endothelial growth factor in regulation of angiogenesis. Antiangiogenic Agents in Cancer Therapy, edited by Teicher BA, Humana Press, New Jersey, pp 119-141.
33. Wang G L, Semenza G L (1993) Desferrioxamine induces erythropoietin gene expression and hypoxia-inducible factor 1 DNA-binding activity: implications for models of hypoxia signal transduction. Blood 82, 3610-3615.
34. Salnikow K, Su W, Blagosklonny M V, Costa M (2000) Carcinogenic metals induce hypoxia-inducible factor-stimulated transcription by reactive oxygen species-independent mechanism. Cancer Res 60, 3375-3378.
35. Bruick R K (2000) Expression of the gene encoding the proapoptotic Nip3 protein is induced by hypoxia. Proc Natl Acad Sci USA 97, 9082-9087.
36. Sowter H M, Ratcliffe P J, Watson P, Greenberg A H, Harris A L (2001) HIF-1-dependent regulation of hypoxic induction of the cell death factors BNIP3 and NIX in human tumors. Cancer Res 61, 6669-6673.
37. Cramer T, Yamanishi Y, Clausen B E, Förster I, Pawlinski R, Mackman N, Haase VH, Jaenisch R, Corr M, Nizet V, Firestein G S, Gerber H-P, Ferrara N, Johnson R S (2003) HIF-1a Is Essential for Myeloid Cell-Mediated Inflammation. Cell 112, 645-657.
38 D'Amico D J (1994) “Diseases of the retina” N. Engl. J. Med. 331:95-106.
39. Aiello L P, Gardner T W, King G L, Blankenship G (1998) “Diabetic retinopathy” Diabetes Care 21:143-56.
40. National Eye Institute (1999) “Vision research—a national plan: 1999-2003” National Eye Institute, National Institute of Health.
41. Aiello L P (1997a) “Clinical implications of vascular endothelial growth factor in proliferative retinopathies” Curr. Opin. Ophthalmol. 8:19-31.
42. Aiello LP (1997b) “Vascular endothelial growth factor and the eye: biochemical mechanisms of action and implications for novel therapies” Ophthalmic Res. 29:354-63.
43. Miller J W (1997) “Vascular endothelial growth factor and ocular neovascularization” Am. J. Pathol. 151:13 23.
44. Aiello L P, Avery R L, Arrigg P G, Keyt B A, Jampel H D, Shah S T, Pasquale L R, Thieme H, Iwamoto M, Park J E, Nguyen H V, Aiello L M, Ferrara N, King G L (1994) “Vascular endothelial growth factor in ocular fluid of patients with diabetic retinopathy and other retinal dosorders” N. Engl. J. Med. 331:1480-7.
45. Ambati J, Chalam K V, Chawla D K, D'Angio C T, Guillet E G, Rose S J, Vanderlinde R E, Ambati B K (1997) “Elevated gamma-aminobutyric acid, glutamate, and vascular endothelial growth factor levels in the vitreous of patients with proliferative diabetic retinopathy” Arch. Ophthalmol. 115:1161-6.
46. Armstrong D, Augustin A J, Spengler R, Al-Jada A, Nickola T, Grus F, Koch F (1998) “Detection of vascular endothelial growth factor and tumor necrosis factor alpha in epiretinal membranes of proliferative diabetic retinopathy, proliferative vitreoretinopathy and macular pucker” Ophthalmologica 212:410-4.
47. Boulton M, Foreman D, Williams G, McLeod D (1998) “VEGF localisation in diabetic retinopathy” Br. J. Ophthalmol. 82:561-8.
48. Cao J, Mathews M K, McLeod D S, Merges C, Hjelmeland L M, Lutty G A (1999) “Angiogenic factors in human proliferative sickle cell retinopathy” Br. J. Ophthalmol. 83:838-46.
49. Pe'er J, Shweiki D, Itin A, Hemo I, Gnessin H, Keshet E (1995) “Hypoxia-induced expression of vascular endothelial growth factor by retinal cells is a common factor in neovascularizing ocular diseases” Lab. Invest. 72:638-45.
50. Kvanta A, Algvere P V, Berglin L, Seregard S (1996) “Subfoveal fibrovascular membranes in age-related macular degeneration express vascular endothelial growth factor” Invest. Ophthalmol. Vis. Sci. 37:1929 34.
51. Lopez P F, Sippy B D, Lambert H M, Thach A B, Hinton D R (1996) “Transdifferentiated retinal pigment epithelial cells are immunoreactive for vascular endothelial growth factor in surgically excised age-realted macular degeneration-related choroidal neovascular membranes” Invest. Ophthalmol. Vis. Sci. 37:855-68.
52. Kliffen M, Sharma H S, Mooy C M, Kerkyliet S, de Jong P T V M (1997) “Increased expression of angiogenic growth factors in age-related maculopathy” Br. J. Ophthalmol. 81:154-62.

1. A compound of the following formula: 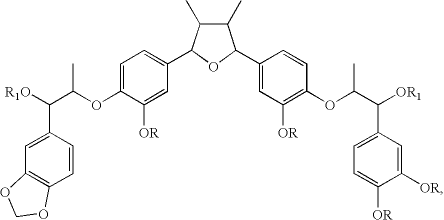
wherein R and R₁ are each the same or different and are independently H, alkyl, acetyl, amine, amide, cyano, thiocyano, aldehyde, halogen, ester, ether, sulfate, carbonate, acetonide, aldehyde, halides, cyano, thiocyano; provided that each R₁ is not H at the same time each R is a methyl; and analogs, stereoisomers, and pharmaceutical salts thereof.

2. A compound of claim 1, of the following formula: 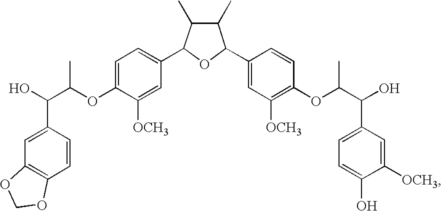
and analogs, stereoisomers, and pharmaceutical salts thereof.

3. A compound of claim 1, of the following formula: 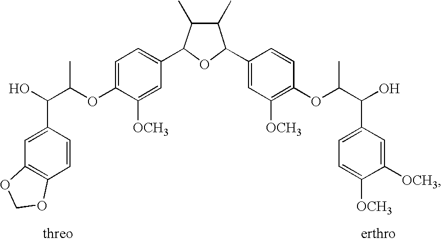
and pharmaceutical salts thereof.

4. A compound of the following formula: 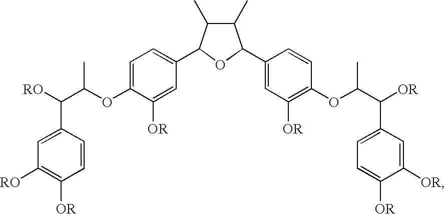
wherein R and R₁ are each the same or different and are independently H, alkyl, acetyl, amine, amide, cyano, thiocyano, aldehyde, halogen, ester, ether, sulfate, carbonate, acetonide, aldehyde, halides, cyano, thiocyano; provided that each R₁ is not H at the same time each R is a methyl; and analogs, stereoisomers, and pharmaceutical salts thereof.

5. A compound of the following formula: 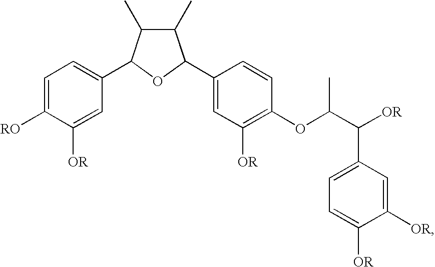
wherein each R is the same or different and independently H, alkyl, acetyl, amine, amide, cyano, thiocyano, aldehyde, halogen, ester, ether, sulfate, carbonate, acetonide, aldehyde, halides, cyano, thiocyano; provided that each R is not a methyl; and analogs, stereoisomers, and pharmaceutical salts thereof.

6. A method of inhibiting HIF-1 function in a subject in need thereof, comprising administering to said subject an effective inhibiting amount of a compound chosen from: 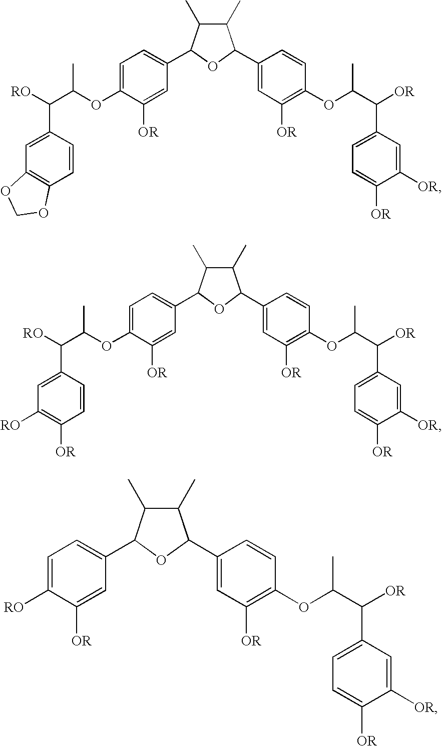
wherein each R is the same or different and independently H, alkyl, acetyl, amine, amide, cyano, thiocyano, aldehyde, halogen, ester, ether, sulfate, carbonate, acetonide, aldehyde, halides, cyano, thiocyano.

7. The method of claim 6, wherein the HIF-1 inhibition ameliorates or prevents indications related to cancer, stroke, heart disease, ocular neovascular disease, arthritis, psoriasis, diabetic retinopathy, macular degeneration.

8. A method of preventing or treating ischemic tissue damage, comprising administering to a patient in need thereof an effective amount of a compound chosen from: 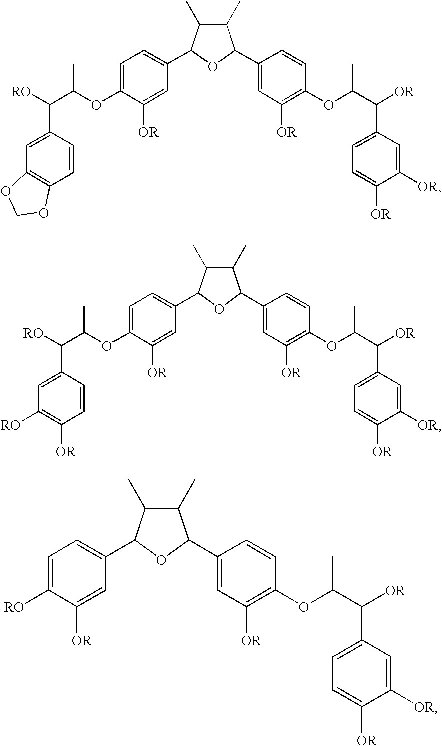
wherein each R is the same or different and independently H, alkyl, acetyl, amine, amide, cyano, thiocyano, aldehyde, halogen, ester, ether, sulfate, carbonate, acetonide, aldehyde, halides, cyano, thiocyano.

9. A method of treating or preventing cancer, comprising administering an effective amount to a subject in need thereof a compound chosen from: 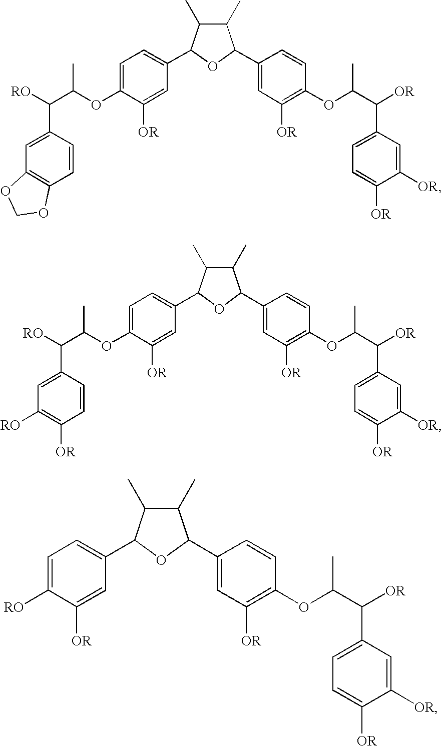
wherein R and R₁ are each the same or different and are independently H, alkyl, acetyl, amine, amide, cyano, thiocyano, aldehyde, halogen, ester, ether, sulfate, carbonate, acetonide, aldehyde, halides, cyano, thiocyano; provided that each R₁ is not H at the same time each R is a methyl; and analogs, stereoisomers, and pharmaceutical salts thereof.

10. The method of claim 9, wherein the treatment is in conjunction with a radiation or chemotherapy cancer treatment.

11. The method of claim 9, wherein the cancer is liver cancer, breast cancer, cervical cancer, esophageal cancer, tongue cancer, oral cancer, pancreas cancer, thyroid cancer, leukemia, myeloma.

12. A method of inhibiting vascular endothelial growth factor (VEGF), comprising administering an effective amount of to a subject in need thereof of a compound chosen from: 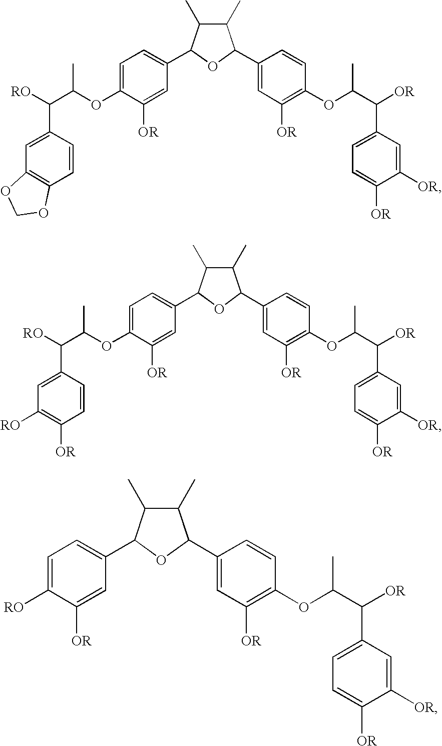
wherein each R is the same or different and independently H, alkyl, acetyl, amine, amide, cyano, thiocyano, aldehyde, halogen, ester, ether, sulfate, carbonate, acetonide, aldehyde, halides, cyano, thiocyano.

13. A method of inhibiting HIF-1 function in a subject in need thereof, comprising:
providing an extract isolated from Saururus cernuus L;
administering an effective HIF-1 function inhibiting amount to the subject.

14. The method of claim 13, wherein the administered amount is in crude extract form.

15. The method of claim 13, wherein the administered amount is in the form of a composition.

16. The method of claim 13, wherein the extract comprises at least one of a compound chosen from:

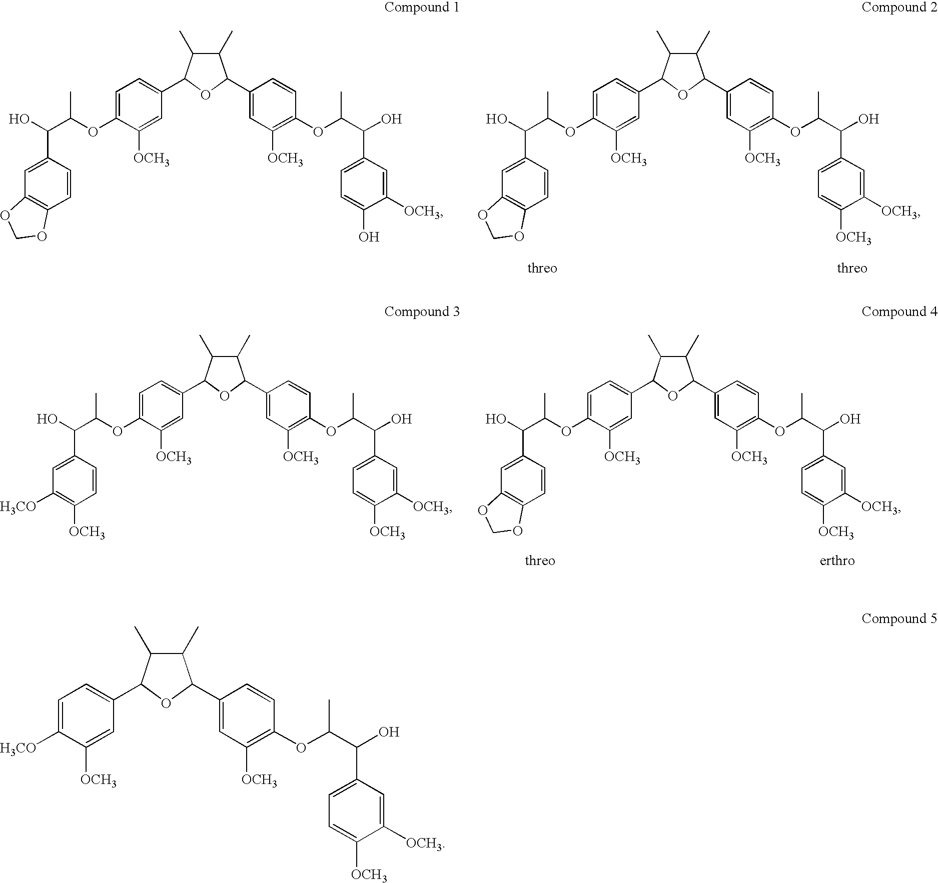

17. The method of claim 13, wherein the HIF-1 inhibition ameliorates or prevents indications related to cancer, stroke, heart disease, ocular neovascular disease, arthritis, psoriasis, diabetic retinopathy, macular degeneration.

18. The method of claim 13, wherein said extract is an alcohol-solvent extract.
